(19) United States
(12) Patent Application Publication
(10) Pub. No.: US 2002/0090664 A1
(43) Pub. Date: Jul. 11, 2002

(54) Methods for measuring stress in mammals

(76) Inventors: Benjamin Wiegand, Newton, PA (US); Laura McCulloch, Cedar Knolls, NJ (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/012,626

(22) Filed: Dec. 7, 2001

Related U.S. Application Data
(60) Provisional application No. 60/256,812, filed on Dec. 20, 2000.

(51) Int. Cl. G01N 33/53; G01N 33/537; G01N 33/543

(52) U.S. Cl. 435/7.92

(57) ABSTRACT

This invention relates to methods for measuring the stress level of a mammal by measuring the activity of the hypothalamus-adrenal system using levels of free salivary adrenocortical hormone as an index of an individuals stress level.

This application claims priority to U.S. patent application Ser. No. 60/256,812, filed Dec. 20, 2000, the disclosure of which is hereby incorporated by reference.

### FIELD OF THE INVENTION

The invention relates to methods for monitoring the stress level of mammals by measuring the activity of the hypothalamus-pituitary adrenal system.

### BACKGROUND OF THE INVENTION

Advances in technology in the last century have brought benefits to society but have resulted in greater prevalence of stress in the daily lives of people at all levels of society. Our stress response mechanisms have not adapted at the same pace as advancing technology. The effect of stress on health and well being is well documented in “Why Zebra's Don't Get Ulcers—An Updated Guide to Stress, Stress Related Diseases and Coping” by Robert M. Sapolsky, ISBN 0-7167-3210-6 and by George P. Chrousos and Philip W. Gold in “The Concepts of Stress and Stress System Disorders—Overview of Physical and Behavioral Homeostasis”, JAMA, Mar. 4, 1992, Vol. 267, No. 9. For example, it is known that stress can cause or aggravate many conditions including immunosuppression and vulnerability to infectious diseases, gastric conditions, sleep problems, depression, premature birth in expectant mothers, low birth weight, degeneration of brain neurons leading to memory and learning problems, elevated blood pressure, heart complications and stroke due to elevated blood lipid levels and other health complications.

The activity of the mammalian stress response is driven by the region in the brain known as the hypothalamus. Specifically, the hypothalamus drives the production of “stress hormones” including catecholamines and glucocorticoids. The hypothalamus responds to a stressor by activating the sympathetic nerve endings in the adrenal medulla to produce adrenaline. The hypothalamus produces corticotropin-releasing hormone (“CRH”) which acts upon the pituitary to release adrenocorticotrophic hormone (“ACTH”) which in turn acts upon the adrenal cortex to promote the production of cortisol. The CRH and sympathetic systems participate in a positive feedback loop so that activation of one system activates the other. Since increased cortisol secretion is an indication that the hypothalamus-pituitary adrenal (“HPA”) axis has been activated, conversely, a decrease in cortisol secretion would indicate a downregulation of HPA axis activity.

While in the short term, the activation of these physiological responses to stress can have beneficial and even life saving merits, long term stress has negative effects on health JBP-575 and well being. If the physiological response to chronic stress is to lead to elevated production of stress hormones, in effect resetting their basal levels, then it could be hypothesized that sustained reduction of these hormones, namely resetting the basal levels to a lower value, would be beneficial in managing stress and promoting well being. Also, as these hormones act upon each other in a positive feedback loop, downregulation of one system would be expected to downregulate the other. Resetting the basal levels of these stress hormones to a lower value could provide benefits including reduced perceived stress; reduced immunosuppression and vulnerability to infectious diseases; reduced incidence of gastric conditions; reduced incidence of sleep problems; reduced incidence of depression; reduced incidence of premature birth; reduced incidence of low birth weight; reduced incidence of degeneration of brain neurons leading to memory and learning problems; reduced incidence of elevated blood pressure; reduced incidence of heart complications and stroke due to elevated blood lipid levels; reduced deleterious effects on metabolism and reproduction; reduced incidence of abdominal adiposity; reduced contribution to aging; reduced incidence of addictive behaviors; and reduced occurrence of other health and behavioral complications that are caused or aggravated by stress.

A good measure of the reactivity of the HPA axis is a measure of adrenocortical activity. An adrenocortical hormone that can be easily measured is cortisol, which can be found in the blood and the saliva of human beings. Cortisol is produced in the adrenal cortex and is involved in a number of neurological events. Some have found that the level of this hormone rises when an individual is subjected to psychological and/or physiological stress. Kirschbaum, C. & Hellhammer, D. H., “Salivary Cortisol in Psychoendocrine Research: Recent Developments and Applications”; Psychoendocrinology, Vol. 19 No. 4, 1994, pp. 313-333. Methodology to accurately measure this adrenocortical hormone has been developed and refined over the past decade and is now applicable to measure HPA axis activity.

It has been recognized by those skilled in the art that a stressor induces an increase in the level of free cortisol which is detectable in saliva. Reports of elevated salivary free cortisol in response to psychological and physiological stress are reported by Kirschbaum, C. & Hellhammer, D. H., “Salivary Cortisol in Psychoendocrine Research: Recent Developments and Applications”; Psychoneuroenocrinology, Vol. 19 No. 4, 1994 pp. 313 333.

Others have found that when adults are subjected to psychological stress (practicing arithmetic under stressful conditions) that their level of stress can be monitored by their salivary cortisol. Tanizawa, “A Method for the Determination of the Anti-Stress Effects of Fragrances” JP Patent No.11-19076. The same researchers have shown that if the same individuals were exposed to certain fragrances before the stressful event, their level of salivary cortisol levels would not be as high as when they were psychologically challenged without the fragrance. Id. This study showed that not all fragrances were effective at reducing the stress induced release of cortisol. Fragrances with lavender oil or mint oil successfully lowered cortisol levels, while the fragrance with skatole had the opposite effect.

While it is possible to objectively measure someones body temperature, which is a measure of their overall health, there has not been any attempt to objectively measure one's overall stress level. This is surprising as we know that stress plays a major role in a number of different diseases and conditions, both functionally and behaviorally. However, thus far stress has been subjectively measured using questionnaires which usually require a trained psychologist or medical professional to interpret the results. Accordingly, there remains a need for methodologies that can chart and map stress levels of mammals over time which can enable individuals to monitor their stress level without the need for consultation with a medical professional to administer testing and interpret results. The present invention answers this need.

### SUMMARY OF THE INVENTION

It has been discovered that the stress level of a mammal can be measured by measuring the activity of the hypothalamus-pituitary-adrenal system. Accordingly, in one embodiment, the invention relates to a method of monitoring the stress level of a mammal comprising:

(a) establishing a baseline stress value by measuring the activity of the hypothalamus-pituitary-adrenal system of said mammal;

(b) at least about 24 hours after step (a) measuring the activity of the hypothalamus-pituitary adrenal system of said mammal; and

(c) comparing the value obtained in step (b) with the value obtained in step (a).

The activity of the hypothalamus-pituitary adrenal system is measured by measuring at least one of the following: (i) waking adrenocortical hormone; (ii) adrenocortical hormone at any time in the period from about 4 to about 8 hours following morning waking; (iii) total daily free adrenocortical hormone; and (iv) total daily free adrenocortical hormone minus the morning peak,

It has been discovered that the methods according to the invention cam be used to measure the readiness of a mammal for a physical or mental challenge. Accordingly, in another embodiment, the invention relates to a method of measuring a mammals readiness for a physical or mental challenge by measuring the activity of the hypothalamus-pituitary-adrenal system using levels of free salivary adrenocortical hormone as an index of readiness said method comprising the steps of

(a) establishing a baseline stress value by measuring the activity of the hypothalamus-pituitary-adrenal system of said mammal using levels of free salivary adrenocortical hormone;

(b) at least about 24 hours after step (a) measuring the activity of the hypothalamus-pituitary-adrenal system of said mammal; and

(c) comparing the value obtained in step (b) with the value obtained in step (a), wherein an increase of about 10% of free salivary adrenocortical hormone over the value of step (a) indicates improved readiness of an individual for a physical or mental challenge,

wherein the activity of the hypothalamus-adrenal system is measured as described above.

### BRIEF DESCRIPTION OF THE DRAWINGS

FIG. 1 is a graph illustrating the “waking adrenocortical hormone.”

FIG. 2 is a graph illustrating the “adrenocortical hormone in a mammal in the period from about 4 to about 8 hours following morning waking.”

FIG. 3 is a graph illustrating the “total free daily adrenocortical hormone.”

FIG. 4 is a graph illustrating the total free daily adrenocortical hormone minus the morning peak.”

### DETAILED DESCRIPTION OF THE INVENTION

As discussed above, the methods according to the invention provide a method in which the stress level of a mammal can be monitored over time without the need for consultation with a medical professional to administer testing and interpret results. Specifically, the invention relates to a method of monitoring the stress level of a mammal comprising:

(a) establishing a baseline stress value by measuring the activity of the hypothalamus-pituitary-adrenal system of said mammal;

(b) at least about 24 hours after step (a) measuring the activity of the hypothalamus-pituitary-adrenal system of said mammal; and

(c) comparing the value obtained in step (b) with the value obtained in step (a).

As used herein, “mammals” include any of a class of warm-blooded higher vertebrates that nourish their young with milk secreted by mammary glands and have skin usually more or less covered with hair, and non-exclusively includes humans, dogs and cats.

As used herein, the term “waking adrenocortical hormone” refers to the total amount of adrenocortical hormone secreted throughout the first hour in the wakeful period of a 24 hour period typically divided into a period of wakefulness and a period of sleepfulness.

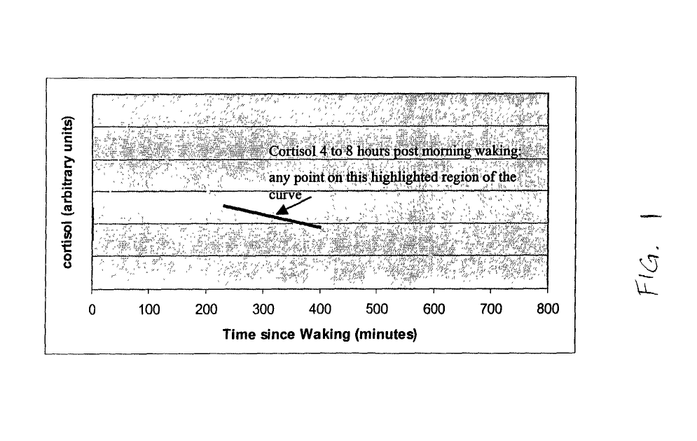

These areas are illustrated for the adrenocortical hormone cortisol in saliva in FIG. 1.

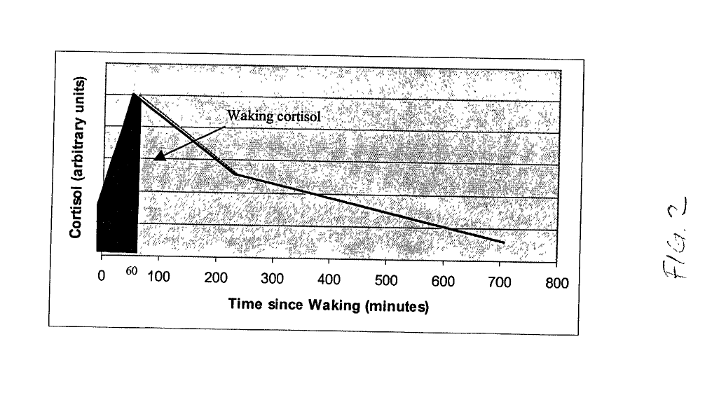

As used herein, the term “adrenocortical hormone in a mammal in the period from about 4 to about 8 hours following morning waking” refers to the amount of adrenocortical hormone secreted at any point in the 4 to 8 hours following morning waking, in any increments of time, for example minutes and hours. Any point on this region of the curve is included in this definition. The region on the curve representing the 4 to 8 hours following morning waking of the adrenocortical hormone cortisol in saliva as a function of time since morning waking is illustrated in FIG. 2.

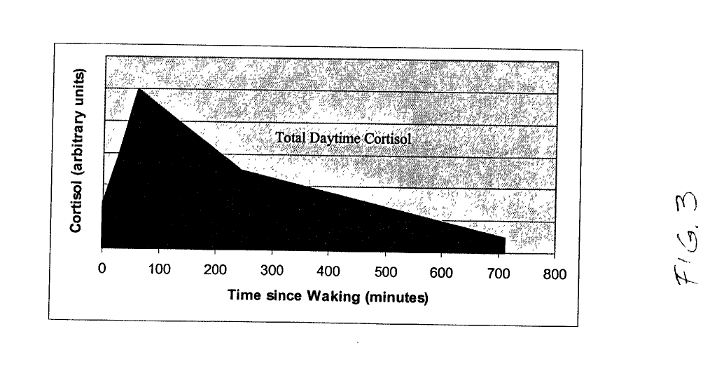

As used herein, the term “total free daily adrenocortical hormone” refers to the total amount of adrenocortical hormone secreted throughout the wakeful period in a 24 hour period typically divided into a period of wakefulness and a period of sleepfulness. The most substantial amount of adrenocortical hormone secreted by an individual during the wakeful period of a 24 hour day is typically secreted in the first 12 hours immediately following morning waking. The area under the curve of salivary cortisol secretion as a function of time since waking for the 12 hour period following morning waking is illustrated in FIG. 3 and is used in examples in this disclosure to represent the total amount of cortisol secreted throughout the wakeful period of a 24 hour day.

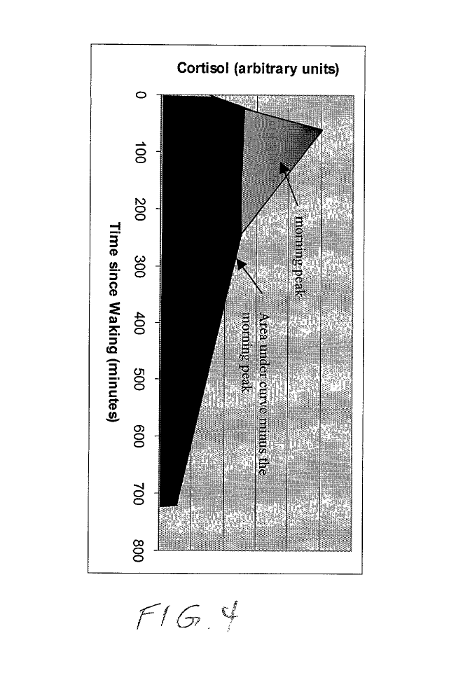

As used herein, the term “total free daily adrenocortical hormone minus the morning peak” refers to the total amount of adrenocortical hormone secreted throughout the wakeful period in a 24 hour period typically divided into a period of wakefulness and a period of sleepfulness, as defined above, having subtracted the area under the morning peak. These areas are illustrated for the adrenocortical hormone cortisol in saliva in FIG. 4.

Cortisol, an adrenocortical hormone, is a good representative marker for adrenocortical activity, and methodology to measure it's level has been developed over the last decade. Cortisol is found in a number of different fluids in the body, including serum, saliva and urine. Recent work done by Hellhammer, et al. has shown that cortisol measures done in saliva samples can be correlated with serum samples and do not have the associated concerns with serum measurements. Firstly, cortisol collection methodology in serum requires either a pinprick, needle, or other device to collect the fluids, which of itself can cause a stressful response. Use of intravenous devices for long term collections are possible, but affect the individuals Quality of Life and are therefore not totally representative of their normal response. Secondly, it is well known that the majority of cortisol in serum is bound to corticosteroid-binding globulin (CBG), albumin and erythrocytes (85% -98%). As it is only the free cortisol that would be expected to impart any physiological effect, it is important to measure this parameter. Urinary cortisol measurements are also possible, however, this would represent a more integrative measure over time, instead of a momentary measure, which is important to better understand the stress profile of the individual. In saliva, much of the cortisol found is free, making this measurement much easier than in serum.

The level of cortisol can be easily measured by taking a saliva sample from the patient, and then performing the appropriate ELISA or RIA methodology as taught, for example, by Kischbaum, C., Hellhammer, D H (1989) Salivary Cortisol in psychobiological research: An Overview, Neuropsychobiology 22: 150-169; Cooper T R, Trunkfield, H R, Zanella A J, Booth, W D (1989) An Enzyme-linked Immunosorbent Assay for Cortisol in the Saliva of Man and Farm Animals. J. Endocrinol 123: R13:R16; and Dressendoerfer, R. A., Kirschbaum, C., Rohde, W., Stahl, F., and Strasburger, C. J. (1992) Synthesis of a Cortisol-Biotin Conjugate and Evaluation as a Tracer in an Immunoassay for Salivary Cortisol Measurement, J. Steroid Biochem. Mo. Biol. 43 683-692, the disclosures of which are hereby incorporated by reference.

Since each person is different in terms of their basal cortisol levels, and their responses to stress, the user must take readings over a single day, to set a baseline for the individual. This information is then captured into an analysis table, which can then be compared against future measurements. In addition, we have found that the time point 4 hours after waking is also a good measure of stress throughout the day, and comparisons on subsequent days against that time point are also useful.

Accordingly, in the method according to the invention, the activity of the hypothalamus-pituitary-adrenal system is measured by measuring at least one of the following: (i waking adrenocortical hormone; (ii) adrenocortical hormone at any time in the period from about 4 to about 8 hours following morning waking; (iii) total daily free adrenocortical hormone; and (iv) total daily free adrenocortical hormone minus the morning peak,

Since there is no “average or normal” stress temperature for every individual, one must first select a day to take a baseline measurement. The choice of the day can be based on any number of reasons, but we would propose two key reasons. First, the day could be chosen, because the individual is “stress” free, e.g. after a vacation, or some restful period. In this case, one is using this invention to measure any increases in stress in the individual. On the other hand, the initial day could be a representative day where the individual has some level of stress. In this case, subsequent measures can be used to determine the amount and effectiveness of a stress management or intervention technique.

On the baseline day, the panelist is instructed to collect a number of saliva samples throughout the day at the prescribed times (upon waking, 30 minutes post waking, 60 minutes post waking, 4 hours post waking, 8 hours post waking, and 12 hours post waking). Prescribed times are selected in order to determine the level of cortisol in saliva throughout the wakeful period of a 24 hour day, and it is obvious to one of ordinary skill in the art, that these times, where possible, should be selected in order to collect a saliva sample which will give the most accurate representation of a panelist's cortisol levels as determined in the subsequent assay procedure. It is also well known to one of ordinary skill in the art that other habits and practices of an individual should be monitored and regulated in order to best achieve accuracy in cortisol level determination.

Samples can be collected throughout the day, for example, prompted by a Palm Pilot, the samples can then be sent to a testing facility. The results can be available, for example, via the internet. In addition, an in situ measurement could be done, and fed into the Palm Pilot. Also, one could look for markers in the person's breath. These samples would then be analyzed for cortisol values at each time point using the appropriate analytical techniques, including but not limited to, ELISA and/or RIA methods discussed above. ELISA methodologies are particularly preferred because they are able to measure cortisol levels in saliva at very low levels. Because the samples do not need to be handled in any special way, and are stable at room temperature for a long period of time, methodologies exist that can now chart and map stress levels of individuals over time. Futhermore as the measure gives the individual an objective measure of their stress level it enables individuals to monitor their stress level without the need for consultation with a medical professional to administer testing and interpret results.

Once these values are obtained, the resulting time course data is used to calculate four different values for the stress “temperature” of the individual. These measurements are waking cortisol, 4 hours after waking cortisol, total daily free cortisol, and total daily free cortisol minus waking cortisol, which have been previously outlined. This set of data serves as a baseline value for the individual.

On a subsequent day of the individual's choice, the same procedure is followed, collecting the saliva samples, having them analyzed for cortisol, and then calculating the four different measures of stress, for a more complete picture. Once these values have been calculated, a comparison can be done between the measured values and the baseline values, to determine the change, if any, in stress “temperature” of the individual from the baseline measurement to the current day. A comparison of all of these values is necessary to help dimensionalize the magnitude of affect one way or the other.

As each of the four measures of HPA activity described above have different sensitivity, it would be expected that a major change in one's stress level should be evidenced by the corresponding changes in a majority of the stress measures. On the other hand, small changes, one way or another in an unconcerted manner, would probably point to experimental error, and subsequent measures on future days should be undertaken to gain a more complete picture of the person's stress level.

The methods according to the invention can be used to monitor the stress level of a mammal, and where appropriate administer a treatment to either reduce or increase the activity of the hypothalamus-pituitary-adrenal system of the mammal.

In cases where it is desired to change the activity of the hypothalamus-pituitary-adrenal system of a mammal, adminsitration of a sensory regimen is suggested. For example, when the difference between the subsequent measure of activity of the hypothalamus-adrenal system, i.e., step (b) and the baseline stress value, i.e., step (a) is at least 5% lower, even 10% greater, the methods according to the invention may comprise an additional step wherein the activity of the hypothalamus-adrenal system is reduced to the original baseline leve.

Accordingly, in another embodiment, the invention relates to relates to a method of regulating the stress level of a mammal comprising:

(a) establishing a baseline stress value by measuring the activity of the hypothalamus-pituitary-adrenal system of said mammal;

(b) at least about 24 hours after step (a) measuring the activity of the hypothalamus-pituitary-adrenal system of said mammal; and

(c) comparing the value obtained in step (b) with the value obtained in step (a);

(d) adjusting the activity of the hypothalamus-pituitary-adrenal system by administration of an effective amount of a regimen of sensory experience.

Examples of a suitable sensory regimen include the administration of sensory stimuli selected from auditory stimuli, visual stimuli, tactile stimuli, gustatory stimuli and olfactory stimuli and combinations thereof.

The term “effective amount” refers to the duration of the regime of sensory experience sufficient to significantly induce a positive modification in the condition to be treated, but low enough to avoid serious side effects (at a reasonable benefit/risk ratio), within the scope of sound medical judgment. The effective amount of the compound or composition will vary with the particular condition being treated, the age and physical condition of the patient being treated, the severity of the condition, the duration of the treatment, the nature of concurrent therapy, the specific compound or composition employed, the particular pharmaceutically-acceptable carrier utilized, and like factors within the knowledge and expertise of the attending physician. For example, the use of smelling a relaxing fragrance and listening to relaxing music 10 minutes, 3 times a day, and taking a bubble bath in the evening, while listening to music, in dim lighting, Use of a multiple sensory regimen can affect the duration that would be needed to create the desired response. Examples of desired responses include reduction in hypothalamus pituitary axis activity and reduction of a total free daily adrenocortical hormone.

If free cortisol is reduced sufficiently and the reduction is sustained over a sufficient period of time, then the quality of life of an individual may be improved.

Using total free daily cortisol (cortisol secreted throughtout the wakeful period in 24 hour period typically divided into a period of wakefulness and a period of sleepfuleness) as an index of HPA activity, total free daily cortisol should be reduced by 5-50% and more preferably by 10-40% and most preferably by 15-30% from the amount secreted on a typical day in which no relaxation regimen has been practised.

Cortisol follows a diurnal rythym with the profile typically exhibiting a morning peak approximately 30 to 45 minutes following waking. The area under the curve of the daytime profile can be considered as comprising 2 areas, the morning peak and the remaining area under curve. These areas are represented in FIG. 1. The area under the curve minus the peak area is yet another useful index of HPA activity. This value should be reduced by 570% and more preferably by 10-60% and most preferably by 20-50% from the amount secreted on a typical day in which no relaxation regimen has been practised.

Another useful index of the activity of the HPA system is the free cortisol level in saliva approximately 4 hours following waking. If this level is sufficiently reduced from it's baseline value then the the quality of life of an individual may be improved. Cortisol 4 hours post waking should be reduced by 5-70% and more preferably by 10-60% and most preferably by 20-50% from the amount secreted on a typical day in which no relaxation regimen has been practised. Stimuli used to provide the sensory experience generally are those which provide an experience which the individual who intends to practice the invention finds pleasant, such as, for example, the regimens described in copending application entitled “Methods For Reducing Stress In Mammals”, filed concurrently herewith, the disclosure of which is hereby incorporated by reference. In another embodiment, stimuli can be provided by the use of the various kits described by copending application entitled “Kit For Reducing Stress”, filed concurrently herewith, the disclosure of which is hereby incorporated by reference.

Examples of stimuli that can be useful in the practice of this invention include, but are not limited to the following: Sensory fragrances, personal care compositions, compact discs, records, tapes, computer software, beverages, such as teas, paintings, murals, books, landscapes, diffuse lighting, videos, movies, meals, music, etc, and combinations thereof.

Suitable fragrances include relaxing fragrances, but are not limited to those relaxing fragrances available from Quest International, an example of which is PD 1861. Also suitable are the fragrances described in copending U.S. patent application Ser. No. 09/676,876, filed Sep. 29, 2000 entitled “Method For Calming Human Beings Using Personal Care Compositions”, the disclosure of which is hereby incorporated by reference.

The sensory fragrance may be produced by blending the selected essential oils and odoriferous components under ambient conditions until the final mixture is homogenous using equipment and methodology commonly known in the art of fragrance compounding. It is preferable to store the final sensory fragrance mixture under ambient conditions for a few hours after mixing before using it as a component of a personal care composition. The personal care compositions of the present invention may then be produced by blending the desired components with the sensory fragrance using equipment and methodology commonly known in the art of personal care product manufacture. In order to improve the solubilization of the sensory fragrance in aqueous personal care compositions, the sensory fragrance may be pre-blended with one or more of the nonionic surfactants. “Personal care compositions” refers to personal cosmetic, toiletry, and healthcare products such as dry and wet wipes, washes, baths, shampoos, gels, soaps, sticks, balms, sachets, pillows, mousses, sprays, lotions, creams, cleansing compositions, powders, oils, bath oils and other bath compositions which may be added to a bath. Personal care compositions may also include, but are not limited to, aerosols, candles, and substances that may be used with vaporizers. The aforementioned wipes, washes, baths, shampoos, gels, soaps, sticks, balms, sachets, pillows, mousses, sprays, lotions, creams, cleansing compositions, oils, bath oils, aerosols, candles and substances which may be used with vaporizers are commercially known to those who have a knowledge of preparing personal care compositions. Suitable personal care composition, include but are not limited to Johnson's Bedtime Bath.

In order to achieve the desired response in a mammal, the personal care composition may be used in a dosing amount that is in accordance with the prescribed directions of the personal care composition.

Although a greater effect is generally achieved when multiple stimuli are used together, it should be obvious to one skilled in the art that a single exposure to an effective stimuli could be envisaged to have the same sustainable effect as multiple exposures to the stimuli described in the body of this invention and so are included in the invention.

As discussed above, it has been discovered, that the administration of a regime of Sensory Experiences can result in a reduction in the stress level of a mammal. It has been previously shown that pharmaceutically active CRH antagonists can provide similar benefits, however, there are resultant side effects that are prevalent when these active materials are used. In another embodiment of the invention, the combination of the use of the sensory regime and the CRH antagonist provides for a more potent treatment. In another embodiment, the combination of the use of the sensory regime and the CRH antagonist, allows for a lower does of the CRH antagonist to be used.

Examples of CRH antagonists include, but are not limited to Astressin, D-PheCRH (12-41), and alpha helical CRH (9-41), and others known in the art. In yet another embodiment, the methods according to the invention may be practiced in combination with the administration of pharmaceuticals that downregulate CRH, such as antidepressants including but not limited to selective serotonin reuptake inhibitors (SSRI), for example Prozac. Such pharmaceuticals should be administered in accordance with the directions prescribed by an authorized physician.

In yet another embodiment of the invention, the invention relates to a method of measuring a mammals readiness for a physical or mental challenge by measuring the activity of the hypothalamus-pituitary-adrenal system using levels of free salivary adrenocortical hormone as an index of readiness said method comprising the steps of

(a) establishing a baseline stress value by measuring the activity of the hypothalamus-pituitary-adrenal system of said mammal using levels of free salivary adrenocortical hormone;

(e) at least about 24 hours after step (a) measuring the activity of the hypothalamus-pituitary-adrenal system of said mammal using levels of free salivary adrenocortical hormone; and

(f) comparing the value obtained in step (b) with the value obtained in step (a), wherein an increase of about 10% of free salivary adrenocortical hormone over the value of step (a) indicates improved readiness of an individual for a physical or mental challenge.

In some cases a treatment or intervention may be recommended to improve the readiness of an individual for a physical or mental challenge by increasing the activity of the hypothalamus-pituitary-adrenal system. Such treatment or intervention may include participating in a regime of stimulation consisting of sensory experiences from the group comprising auditory stimuli, visual stimuli, tactile stimuli, gustatory stimuli, olfactory stimuli and optionally use of a CRH agonist as discussed above.

In order to illustrate the invention the following prophetic examples are included. These examples do not limit the invention. They are meant only to suggest a method of practicing the invention.

### EXAMPLES

### Example 1

A baseline profile of the user's hypothalamus-pituitary-adrenal-axis, and accordingly stress level, is established by instructing the user to collect a series of saliva samples throughout the day for the purpose of measuring free cortisol. Suggested timepoints for the collection of these samples are upon waking, 30 minutes post waking, 60? minutes post waking, 4 hours post waking, 8 hours post waking, and 12 hours post waking.

The saliva samples collected at each of these timepoints are subsequently analyzed for free cortisol concentration using an appropriate analytical technique. The cortisol concentration at each of these timepoints is plotted on the y-axis of a Cartesian graph, in which time since waking is plotted on the x-axis. The data may be represented graphically using appropriate means such as graph paper or software used with desk top, laptop and hand held computers and mobile telecommunication devices.

The data contained in this graph may be used to calculate a series of parameters that are useful in evaluating the HPAA and accordingly the stress level of the user. These parameters have been previously outlined and include waking cortisol, 4 hours after waking cortisol, total daily free cortisol, and total daily free cortisol minus the morning peak.

On a subsequent day of the user's choice, the same procedure is followed: collecting the saliva samples for cortisol analysis, and then calculating the four different measures of stress. Once these values have been calculated, a comparison can be made between the measured values and the baseline values, to determine the change, if any, in stress level of the user from the baseline measurement to the current day.

A comparison of all of these values signifies the magnitude of the increase or decrease in stress level of the user. As each of these four values have different sensitivity, it would be expected that a major change in one's stress level should be evidenced by the corresponding changes in a majority of the stress measures. On the other hand, small changes, in either direction in an inconsistent manner, would probably point to experimental error, and subsequent measures on future days should be undertaken to gain a more complete picture of the person's stress level.

### Example 2

A day in which the user subjectively determines is free of stress, or has only low stress, such as Following a vacation or other restful day, is selected as the baseline day. Baseline adrenocortical hormone, and accordingly stress level, data is collected and calculated as outlined in example 1.

On a subsequent day in which the user is exposed to a stress level greater than that of the baseline day, adrenocortical hormone data is collected. From a comparison of this data with the baseline data, the user has knowledge of the increase in adrenocortical hormone secretion, and accordingly the increase in their stress level. A change of greater than about 5% in the user's adrenocortical hormone parameters would signify an increase in stress level. The significance of the increase in stress level is greatest where all of these parameters indicate an increase as compared to their baseline values.

Upon determining that the user has experienced an increase in stress level, a range of interventions or stress reducing regimens are recommended. The range of recommendations can be presented in the form of a written or electronic list. Further, the recommendations can be tailored to suit the users personal tastes and interests and also to the degree of severity of the stress increase.

To manage a relatively minor increase in stress level, the user may be encouraged, for example to listen to some soothing music appropriate to their personal musical preference. To manage a more significant increase in stress level, the user may be encouraged to visit a medical professional for pharmaceutical or medical intervention that could be accompanied by a sensory regimen selected from the group of olfactory, visual, gustatory, audio and tactile stimuli, and combinations thereof.

### Example 3

A day in which the user subjectively determines is stressful is selected as the baseline day. Baseline adrenocortical hormone, and accordingly stress level, data is collected and collected as outlined in example 1.

A stress management treatment or intervention is selected by the user. The range of treatment or intervention can be presented in the form of a written or electronic list, or may have been prescribed by a medical professional or other professional in the area of stress management.

On subsequent day(s), either during or following the stress management intervention, adrenocortical hormone data is collected and parameters useful in determining the user's stress level are calculated. A comparison of these values with the baseline value is made. A decrease in the adrenocortical hormone parameters indicates that the intervention is effective. A decrease in all of the adrenocortical hormone parameters is indicative of greatest efficacy of the intervention.

### Example 4

While the use of multiple measures and adrenocortical hormone provides the most accurate picture of a user's stress level, single measures of adrenocortical hormone concentration are useful in determining the stress level of an individual. In particular the adrenocortical hormone levels in the 4 to 8 hour period following waking are useful as single measures of adrenocortical hormone level and accordingly stress level.

A user wishing to use the stress thermometer in this way would firstly collect baseline data. This baseline data may be collected on a relatively low stress day, as described in Example 2 or on a more stressful day, as described in Example 3. Single measure may be collected in the 4 to 8 hours following waking along, with a note of the time that had elapsed between morning waking and the time that sample was collected. This value is then recorded as a baseline value that can be compared to values from samples collected at the same timepoint following waking on subsequent days.

A decrease in this value on subsequent days would indicate a reduction in stress, and conversely an increase in this value on subsequent days would indicate an increase in stress level and could be accompanied by a recommended stress management treatment.

More preferably the user will collect baseline data over a full day as outlined in Examples 1-3 above. On subsequent days, the user may collect a single sample, most preferably in the 4 to 8 hour period following waking, along with a note of the time that had elapsed between morning waking and the time that sample was collected. This value will then be compared to the corresponding value on the baseline day curve.

A decrease in this value on subsequent days would indicate a reduction in stress, and conversely an increase in this value on subsequent days would indicate an increase in stress level and could be accompanied by a recommended stress management treatment.

### Example 5

A day in which the user subjectively determines is free of stress, or has only low stress, such as that following a stress treatment or intervention, following a vacation or other restful day, is selected as the baseline day. Baseline adrenocortical hormone, and accordingly stress level, data is collected and calculated as outlined in Example 1.

On a subsequent day in which the user wishes to confirm that their stress level has not changed significantly from that of their stress level on the baseline day, adrenocortical hormone data is collected. From a comparison of this data with the baseline data, the user has knowledge of any increase in adrenocortical hormone secretion, and accordingly any increase in their stress level. A change of less than about 5% in the user's adrenocortical hormone parameters would signify that there had been no significant change in their stress level. The significance of this confirmation of no change in stress level, and that their stress level has been maintained, as compared to the baseline day, is greatest where all of these parameters indicate an increase of no greater than about 5% as compared to their baseline values.

What is claimed is:

1. A method of monitoring the stress level of a mammal comprising:
(a) establishing a baseline stress value by measuring the activity of the hypothalamus-pituitary-adrenal system of said mammal using levels of total free salivary adrenocortical hormone;
(b) at least about 24 hours after step (a) measuring the activity of the hypothalamus-pituitary-adrenal system of said mammal using levels of total free salivary adrenocortical hormone; and
(c) comparing the value obtained in step (b) with the value obtained in step (a).

2. A method according to claim 1, wherein said total free salivary adrenocortical hormone is measured using an ELISA or RIA technique.

3. A method according to claim 1, wherein the value of (b) is at least about 5% greater than the value of (a), further comprising step (d), reducing the activity of the hypothalamus-pituitary-adrenal system of said mammal.

4. A method according to claim 3, wherein said step (d) comprises administering an effective amount of a sensory regimen to said mammal.

5. A method according to claim 4, wherein the sensory regimen is selected from the group consisting of auditory stimuli, visual stimuli, tactile stimuli, gustatory stimuli and olfactory stimuli, and combinations thereof.

6. A method according to claim 5, wherein the regimen further includes the administration of a CRH antagonist or an antidepressant.

7. A method of monitoring the stress level of a mammal comprising:
(a) establishing a baseline stress value by measuring the activity of the hypothalamus-pituitary-adrenal system using total free daily adrenocortical hormone minus the morning peak;
(b) at least about 24 hours after step (a) measuring the activity of the hypothalamus-pituitary-adrenal system using total free daily adrenocortical hormone minus the morning peak; and
(c) comparing the value obtained in step (b) with the value obtained in step (a).

8. A method according to claim 7, wherein said free daily adrenocortical hormone minus the morning peak is measured using an ELISA or RIA technique.

9. A method according to claim 7, wherein the value of (b) is at least about 5% greater than the value of (a), further comprising step (d), reducing the activity of the hypothalamus-pituitary-adrenal system of said mammal.

10. A method according to claim 9, wherein said step (d) comprises administering an effective amount of a sensory regimen to said mammal.

11. A method according to claim 10, wherein the sensory regimen is selected from the group consisting of auditory stimuli, visual stimuli, tactile stimuli, gustatory stimuli and olfactory stimuli, and combinations thereof.

12. A method according to claim 11, wherein the regimen further includes the administration of a CRH antagonist or an antidepressant.

13. A method of monitoring the stress level of a mammal comprising:
(a) establishing a baseline stress value by measuring the levels of free salivary adrenocortical hormone in the period of from about 4 to about 8 hours following morning waking;
(b) at least about 24 hours after step (a) the levels of free salivary adrenocortical hormone in the 4-8 hours following morning waking; and
(c) comparing the value obtained in step (b) with the value obtained in step (a).

14. A method according to claim 13, wherein said free salivary adrenocortical hormone is measured using an ELISA or RIA technique.

15. A method according to claim 13, wherein the value of (b) is at least about 5% greater than the value of (a), further comprising step (d), reducing the activity of the hypothalamus-pituitary-adrenal system of said mammal.

16. A method according to claim 15, wherein said step (d) comprises administering an effective amount of a sensory regimen to said mammal.

17. A method according to claim 16, wherein the sensory regimen is selected from the group consisting of auditory stimuli, visual stimuli, tactile stimuli, gustatory stimuli and olfactory stimuli, and combinations thereof.

18. A method according to claim 17, wherein the regimen further includes the administration of a CRH antagonist or an antidepressant.

19. A method of monitoring the stress level of a mammal comprising:
(a) establishing a baseline stress value by measuring the level of free salivary adrenocortical hormone 4 hours following morning waking;
(b) at least about 24 hours after step (a) the levels of free salivary adrenocortical hormone 4 hours following morning waking; and
(c) comparing the value obtained in step (b) with the value obtained in step (a).

20. A method according to claim 19, wherein said free salivary adrenocortical hormone is measured using an ELISA or RIA technique.

21. A method according to claim 19, wherein the value of (b) is at least about 5% greater than the value of (a), further comprising step (d), reducing the activity of the hypothalamus-pituitary-adrenal system of said mammal.

22. A method according to claim 21, wherein said step (d) comprises administering an effective amount of a sensory regimen to said mammal.

23. A method according to claim 22, wherein the sensory regimen is selected from the group consisting of auditory stimuli, visual stimuli, tactile stimuli, gustatory stimuli and olfactory stimuli, and combinations thereof.

24. A method according to claim 23, wherein the regimen further includes the administration of a CRH antagonist or an antidepressant.

25. A method of monitoring the stress level of a mammal comprising:
(a) establishing a baseline stress value by measuring the levels of waking adrenocortical hormone in the first hour following morning waking;
(b) at least about 24 hours after step (a) the levels of waking salivary adrenocortical hormone in the first hour following morning waking; and
(c) comparing the value obtained in step (b) with the value obtained in step (a).

26. The method of claim 25, wherein free salivary adrenocorticol hormone is measured using an ELISA or RIA technique.

27. A method according to claim 25, wherein the value of (b) is at least about 5% greater than the value of (a), further comprising step (d), reducing the activity of the hypothalamus-pituitary-adrenal system of said mammal.

28. A method according to claim 27, wherein said step (d) comprises administering an effective amount of a sensory regimen to said mammal.

29. A method according to claim 28, wherein the sensory regimen is selected from the group consisting of auditory stimuli, visual stimuli, tactile stimuli, gustatory stimuli and olfactory stimuli, and combinations thereof.

30. A method according to claim 29, wherein the regimen further includes the administration of at least one of a CRH antagonist or an antidepressant.

31. A method of measuring the readiness of a mammal for a physical or mental challenge by measuring the activity of the hypothalamus-pituitary-adrenal system using levels of free salivary adrenocortical hormone as an index of readiness said method comprising the steps of
(a) establishing a baseline stress value by measuring the activity of the hypothalamus-pituitary-adrenal system of said mammal using levels of free salivary adrenocortical hormone;
(b) at least about 24 hours after step (a) measuring the activity of the hypothalamus-pituitary-adrenal system of said mammal using levels of free salivary adrenocortical hormone; and
(c) comparing the value obtained in step (b) with the value obtained in step (a), wherein an increase of about 10% of free salivary adrenocortical hormone over the value of step (a) indicates improved readiness of an individual for a physical or mental challenge.

32. The method of claim 31, wherein a treatment or intervention is recommended to improve the readiness of an individual for a physical or mental challenge by increasing the activity of the hypothalamus-pituitary-adrenal system.

33. The method of claim 32, wherein the recommended treatment or intervention may include participating in a regime of stimulation consisting of sensory experiences from the group comprising auditory stimuli, visual stimuli, tactile stimuli, gustatory stimuli, olfactory stimuli and optionally use of a CRH atagonist.

34. A method of monitoring, resetting and maintaining the stress level of a mammal comprising:
(a) establishing a baseline stress value by measuring the activity of the hypothalamus-pituitary-adrenal system of said mammal using levels of total free salivary adrenocortical hormone;
(b) at least about 24 hours after step (a) measuring the activity of the hypothalamus-pituitary-adrenal system of said mammal using levels of total free salivary adrenocortical hormone; and
(c) comparing the value obtained in step (b) with the value obtained in step (a).
(d) administering a treatment regimen to downregulate the activity of the hypothalamus-pituitary-adrenal system of said mammal using levels of total free salivary adrenocortical hormone;
(e) at least about 24 hours after step (a) measuring the activity of the hypothalamus-pituitary-adrenal system of said mammal using levels of total free salivary adrenocortical hormone; and
(f) comparing the value obtained in step (b) with the value obtained in step (a).

35. A method according to claim 34, wherein said total free salivary adrenocortical hormone is measured using an ELISA or RIA technique.

36. A method according to claim 34, wherein the value of (b) is at least about 5% greater than the value of (a), further comprising step (g), reducing the activity of the hypothalamus-pituitary-adrenal system of said mammal.

37. A method according to claim 36, wherein said step (g) comprises administering an effective amount of a sensory regimen to said mammal.

38. A method according to claim 37, wherein the sensory regimen is selected from the group consisting of auditory stimuli, visual stimuli, tactile stimuli, gustatory stimuli and olfactory stimuli, and combinations thereof.

39. A method according to claim 38, wherein the regimen further includes the administration of a CRH antagonist, or antidepressants including but not limited to SSRI's.
